(19) United States
(12) Patent Application Publication
(10) Pub. No.: US 2004/0220672 A1
(43) Pub. Date: Nov. 4, 2004

(54) Orthopedic implants, methods of use and methods of fabrication

(76) Inventor: John H. Shadduck, Tiburon, CA (US)

Correspondence Address: John H. Shadduck, 1490 Vistazo West, Tiburon, CA, 94920, US

(21) Appl. No.: 10/837,858

(22) Filed: May 3, 2004

Related U.S. Application Data
(60) Provisional application No. 60/467,440, filed on May 3, 2003.

(51) Int. Cl. A61F 2/44; A61F 2/28

(52) U.S. Cl. 623/17.16; 623/23.58

(57) ABSTRACT

An orthopedic implant device that is adapted for providing a support structure in spine treatments and other bone treatments. In several embodiments, an exemplary implant body is capable of a first compacted shape and a second expanded shape. The implant is microfabricated of an open cell elastomeric shape memory polymer (SMP) composition. The implant has a first shape for deployment with the SMP in its temporary compacted shape. When deployed in an orthopedic space, the polymer monolith transforms to its memory shape to occupy the space. In several embodiments, the open cell implant body is then infused with an in-situ polymerizable composition to create a composite resilient, fiber-reinforced implant. In another embodiment, the implant body is in-filled with a bone-cement such as PMMA to fill a cavity in a bone, wherein the elastomeric open cell monolith deforms to provide a substantially fluid-impermeable surface around the bone cement.

### CROSS-REFERENCE TO RELATED APPLICATIONS

This application claims benefit of Provisional U.S. Patent Application Ser. No. 60/467,440 filed May 3, 2003 titled Dynamic Spine Stabilization Implants, Methods of Use and Method of Fabrication, and this application is related to the following U.S. Patent Applications: Ser. No. 60/448,498 filed Feb. 18, 2003 titled Intervertebral Disc Implants, Methods of Use and Method of Fabrication; Ser. No. 60/438,352 filed Jan. 7, 2003 titled Medical Implant Devices, Methods of Use and Methods of Fabrication and Ser. No. 60/436,296 filed Dec. 23, 2002 titled Disc Implant Devices of Elastic Composites, all of which are incorporated herein by this reference.

### BACKGROUND OF THE INVENTION

1. Field of the Invention

The invention relates generally to polymeric spinal implant devices and methods, and more particularly relates to microfabricated open cell shape memory polymer structures for implanting in a space in spine structure for used in conjunction with an in-situ polymerizable infill component to create a composite support structure for treating a spine abnormality. In an exemplary embodiment, the composite implant occupies a space in a disc nucleus to increase the intervertebral spacing and foraminal height to reduce discogenic pain.

2. Description of the Prior Art

Lumbar spinal disorders and discogenic pain are major socio-economic concerns in the United States affecting over 70% of the population at some point in life. Low back pain is the most common musculoskeletal complaint requiring medical attention; it is the fifth most common reason for all physician visits. The annual prevalence of low back pain ranges from 15% to 45% and is the most common activity-limiting disorder in persons under the age of 45. Degenerative changes in the intervertebral disc (UVD) play a principal role in the etiology of low back pain.

Many surgical and non-surgical treatments exist for patients with degenerative disc disease (DDD), but often the outcome and efficacy of these treatments are uncertain. The traditional treatment for discogenic low back pain is fusion of the painful vertebral motion segment, for patients that have not found relief from chronic pain through conservative treatments. In the United States, over 200,000 spinal fusion surgeries are performed each year. While there have been significant advances in spinal fusion devices and surgical techniques, the procedure does not always work reliably. In one survey, the average clinical success rate for pain reduction was about 75%; and long time intervals were required for healing and recuperation (3-24 months, average 15 months). Probably the most significant drawback of spinal fusion is termed the “transition syndrome” which describes the premature degeneration of discs at adjacent levels of the spine. This is certainly the most vexing problem facing relatively young patients when considering spinal fusion surgery.

More recently, technologies have been proposed or developed for disc replacement and regeneration that may replace, in part, the role of spinal fusion. One form of complete artificial disc implant has been used in Europe and is currently being tested in clinical trials in the United States. The principal advantage proposed by complete artificial discs is that vertebral motion segments will retain some degree of motion at the disc space that otherwise would be immobilized in more conventional spinal fusion techniques.

Other implant systems have been developed that replace only the disc's inner nucleus with various hydrogels, polymers, inflatable structures and the like that utilize the natural annular lining (annulus fibrosus) of the disc to contain the nucleus implant. One prior art disc nucleus implant is the PDN-SOLO™ prosthetic disc nucleus manufactured by Raymedica, Inc., 9401 James Avenue South, Suite 120, Minneapolis, Minn. 55431. Patents related to the Raymedica disc implant are U.S. Pat. Nos. 5,674,295; 5,824,093; 6,022,376 and 6,132,465.

Other treatments in the investigative stage relate to the introduction of genetically-engineered cells into a degenerated disc, in theory, to regenerate disc material so that its functionality is restored. There is limited experience with the use surgically implanted or injected bioengineered cells in the reproduction of knee cartilage, so there is a long-term possibility that such technologies will prove useful in the spine.

The intervertebral disc (IVD) is a mechanically complex and biologically active system. In terms of mechanical aspects, the intervertebral disc (IVD) supports large loads and permits multi-axial motions of the spine with three essential mechanical functions, including functioning as a spacer, as a shock absorber, and as a motion unit. First, as a spacer, the height of the disc maintains the separation distance between the adjacent vertebral bodies. This allows biomechanics of motion to occur, with the cumulative effect of each spinal segment yielding the total range of motion of the spine in any of several directions. Such proper spacing also is important because it allows the intervertebral foramen to maintain its height, which provides space for the segmental nerve roots to exit each spinal level without compression (i.e., a pinched nerve). Second, the disc functions to absorb shocks to allow the spine to compress and rebound when axially loaded during physical activities. Also, the IVDs collectively resist the downward pull of gravity on the head and trunk during prolonged sitting and standing. Third, the elasticity of the discs allows motion coupling, so that the spinal segments may flex, rotate, and bend to the side all at the same time during a particular activity. This would be impossible if each spinal segment were locked into a single axis of motion.

The intervertebral disc (IVD) consists of several anatomic zones: (i) the outer annulus fibrosus AF; (ii) the transition zone, a thin zone of fibrous tissue between inner annulus and the nucleus pulposus, and (iii) the core gel-like nucleus pulposus NP. The annulus fibrosus AF is a laminated fiber composite with collagen fibers in alternating oblique layers between adjacent vertebrae. The annulus fibrosus AF and the cartilaginous endplates CE contain the nucleus pulposus NP laterally and superiorly/inferiorly (see FIG. 1).

The annulus fibrosus AF is an outer ligamentous ring around the nucleus pulposus that hydraulically seals the nucleus to thereby allow intradiscal pressures to rise as the disc is loaded. The annulus consists of 10 to 20 concentric lamellae of collagen fibers angled relative to horizontal plane of the disc. The lamellae of the outer part of the annulus fibrosus are attached to the ring apophysis of the adjacent upper and lower vertebral bodies. The inner lamellae of the annulus fibrosus are attached to the vertebral endplates. The architecture of the annulus fibrosus AF allows torsional stresses to be distributed through the annulus under normal loading without rupture. The annulus fibrosus similarly re-distributes loads under tension and shear loading. The gelatinous nucleus pulposus NP is largely water, with its solid portions being Type II collagen and non-aggregated proteoglycans (PG). The disc thus functions thus somewhat like a hydraulic cylinder. The annulus interacts with the nucleus, so that when the nucleus is pressurized by vertical loads, the annulus fibers serve in a containment function to prevent the nucleus from bulging the annulus or herniating. The gelatinous nuclear material directs the forces of axial loading outward, and the annular fiber lamellae distribute the force without injury. In-vivo, the IVD is daily subjected to large axial compressive forces-ranging up to several body weights in even the most modest physical activities.

In terms of biological aspects, the IVD can be described as a biologic osmotic pressure system. The nucleus pulposus NP consists of a central core of a well-hydrated PG matrix entrapped in a loose, irregular meshwork of collagen fibers. The high content of proteoglycans in the nucleus pulposus NP causes the disc to imbibe water, which allows the disc to maintain its height and loading bearing capacity to serve both as a spacer and shock absorber. Since the IVD is avascular with low oxygen tension, diffusion is the principal path of nutrient delivery within the disc, and a resulting acidic pH create a biologically severe environment-especially in the nucleus pulposus NP.

In a child or young adult, water accounts for over 80% of the weight of the nucleus pulposus. The water-retaining ability of the nucleus pulposus progressively degrades age. The mechanical properties of the nucleus are associated with the degree of proteoglycan deterioration therein, and the consistency of the nuclear material undergoes a change into clumps rather than being a homogenous material with aging. Such clumping leads to the altered distribution of pressures within the disc and resistance to the flow of nuclear material, which becomes mechanically unstable. At the lateral aspect of the posterior longitudinal ligament, the nucleus clumps can herniate through a weakened region of the annulus and into the spinal canal or foramen.

Further dehydration of the nucleus occurs as the hyaluronic long chains shorten, and decreased swelling pressure results from such deterioration. The degeneration and dehydration of the disc produces micromotion instability and also can cause leakage of nucleus pulposus proteins out of the disc space to inflame innervated structures. The dehydration and altered mechanical stiffness of the nucleus causes the annulus and redundant annular ligaments be compressed with a corresponding loss of disc and foramina height. With progressive nuclear dehydration, the annular fibers can also tear. Loss of normal soft tissue tension may allow the spinal segment to sublux (e.g. partial dislocation of the joint), leading to further foraminal narrowing, mechanical instability, and pain. Often times, a twisting injury can damage the disc and start a cascade of events that leads to disc degeneration. Thus, the natural aging process or trauma can cause disc degeneration, which results in low back pain.

The nucleus pulposus contains large quantities of very inflammatory proteins. Nerves within the disc space only penetrate into the very outer region of the annulus fibrosus. Even though there is little enervation in the annulus, it too can become a significant source of pain if a tear in the annulus allows inflammatory proteins to reaches the outer disc regions where nerves become sensitized. If disc degeneration results in radial tears and leakage from the nuclear material that contacts a nerve root, the resulting inflammatory response will create pain within the patient's leg (sciatica or a radiculopathy). Macrophages then respond to the displaced foreign material and clear the spinal canal. Subsequently, a significant scar can be produced that can result in acute neural compression that causes further dysfunction. For example, compression of a motor nerve can result in limb weakness and sensory nerve compression results in numbness. Disc deterioration and loss of disc height also shifts the balance of weight bearing to the facet joint. This mechanism is believed to be a cause of pain through the facet joint capsule, as well as other tissues attached to and between the posterior bony elements. The disc itself has no blood supply, and hence lacks any significant reparative powers. Since the disc cannot repair itself, pain created by the degenerated disc can last for years.

Clinical stability in the spine can be defined as the ability of the spine under physiologic loads to limit patterns of displacement so as to not damage or irritate the spinal cord or nerve roots. In addition, such clinical stability will prevent incapacitating deformities or pain due to later spine structural changes. Any disruption of the components that stabilized a vertebral segment (i.e., ligaments, disc, facets) decreases the clinical stability of the spine.

Improved methods and techniques are needed for treating dysfunctional intervertebral discs to provide clinical stability, in particular: (i) implantable devices that can be introduced replace a disc nucleus through least invasive procedures; (ii) nucleus implants that can restore disc height and foraminal spacing without damaging the architecture of the annulus fibrosus; and (iii) nucleus implants that can re-distribute loads within disc space in spine flexion, extension, lateral bending and torsion.

### BRIEF DESCRIPTION OF THE DRAWINGS

The features and advantages of this invention, and the manner of attaining them, will become apparent by reference to the following description of preferred embodiments of the invention taken in conjunction with the accompanying drawings, wherein:

FIG. 1 is a schematic view of an intervertebral space with the outline of a nucleus implant body corresponding to the invention together with an indication of access to the site, the implant body comprising at least in part a shape memory polymer material.

FIG. 2A is a sectional view of adjacent vertebrae with a schematic view of the sectional shape of an exemplary implant body.

FIG. 2B is similar to FIG. 2A with an alternative sectional shape of an implant body.

FIG. 3 is a cut-away view of an exemplary nucleus implant of an open cell shape memory polymer (SMP), with endplate and core SMP portions for creating different open percentages and rubbery moduli.

FIG. 4 illustrates a greatly enlarged schematic view of a portion of a soft lithography microfabricated implant body corresponding to the invention.

FIG. 5 illustrates an exploded view of implant body of FIG. 3 with interior SMP portions to provide a selected gradient rubbery modulus.

FIG. 6 illustrates an exploded view of an alternative implant body similar to that of FIG. 5.

FIG. 7 illustrates an alternative implant body with a plurality of interior SMP portions to provide a gradient asymmetric rubbery modulus.

FIG. 8 illustrates an alternative implant body with a plurality of interior SMP portions to provide an asymmetric rubbery modulus.

FIGS. 9A-9C illustrates a method of the invention in introducing the SMP implant component of FIG. 3 into a space created within a disc nucleus, with FIG. 9A illustrating the implant body in its temporary compacted shape and FIGS. 9B-9C illustrating then implant body expanding to its memory shape.

FIG. 10A is a view of an alternative SMP implant body in its temporary compacted shape being introduced into a space for treating an annulus region, wherein the implant body carries a reinforcing material.

FIG. 10B is a view of the SMP implant body of FIG. 10A after expansion to its memory shape to position the reinforcing material against a defect in the targeted annulus region.

FIG. 11 is a schematic view of an alternative open cell implant monolith for treating a bone abnormality.

FIG. 12 is a view of the implant monolith of FIG. 11 with the injection of a bone cement into the interior of the implant.

### DETAILED DESCRIPTION OF THE INVENTION

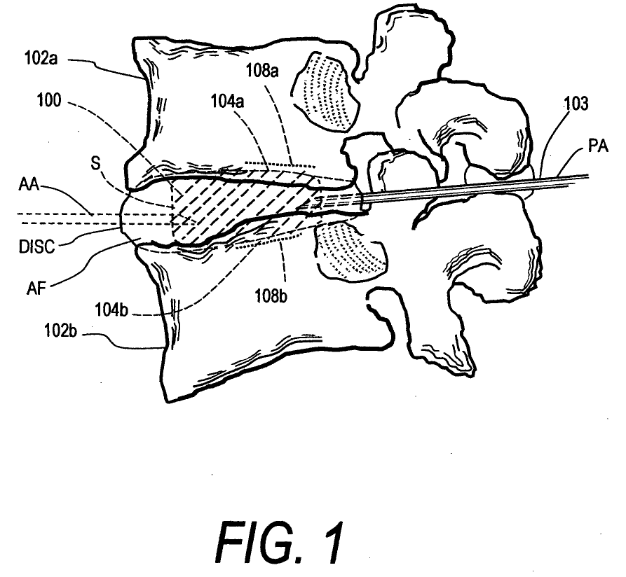

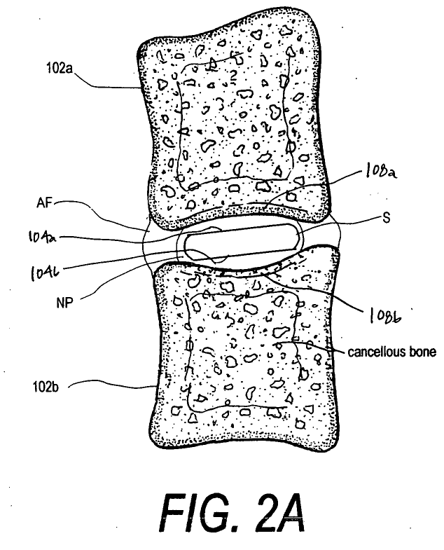

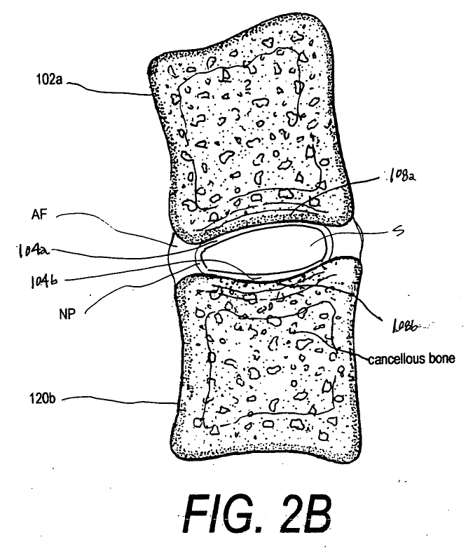

Referring now to FIGS. 1 and 2, an exemplary embodiment of disc nucleus implant 100 corresponding to the invention is shown in phantom and schematic views. As can be understood in FIG. 1, a space S is created within a targeted degenerated disc D with any type of surgical instrument, for example (i) a mechanical cutting and suction/extraction device, (ii) an Rf ablation device, (iii) a laser ablation device, or (iv) another morcellation and extraction system known in the art. Typically, such systems provide an instrument with a distal working end that includes a mechanism for expanding the cross section of the space S being created while maintaining a small diameter entry for the introducer portion. The space S within the disc can be any size, and preferably is substantially circular and approximates the anterior-to-posterior dimension of the native disc nucleus pulposus, or the space can comprise the entire envelope of the native nucleus pulposus. The disc's annulus fibrosus AF is left intact except for the small cross-section access penetration. The scope of the invention extends to any implant sizes, shapes or plurality of partial nucleus implants that use the apparatus and methods described herein.

As can be seen in FIG. 1, the implant 100 is preferably introduced from either of two posterior approaches PA as is known in the art through a minimally invasive incision to the disc between exemplary vertebrae 102a and 102b, for example in the lumbar segment. An introducer 103 is used to deploy the implant 100. The scope of the invention includes an anterior approach indicated at AA in FIG. 1.

FIG. 1 illustrates adjacent vertebrae 102a and 102b in a lumbar segment and indicates that the space S created to receive the implant may have generally convex anterior and posterior surfaces 104a and 104b to engage the vertebral endplates. The superior and inferior aspects of the vertebral space may be more planar in some disc locations. The intervertebral discs are substantially oval in vertical cross-section as shown in FIG. 1, with the height of the discs increasing in varied degrees from the periphery to the center which is defined herein as a bi-convex shape. A longitudinal ligament attaches to the vertebral bodies and to the intervertebral discs anteriorly and posteriorly, and the cartilaginous endplates of each disc is attached adjacent to the bony endplates 108 and 108b of the vertebral bodies.

FIGS. 2A-2B illustrate adjacent vertebrae 102a and 102b in a lumbar segment and a native disc made up of annulus fibrosus AF and nucleus pulposus NP. The nucleus has a space S created therein to receive the implant. The space may have a vertical cross-section with planar surfaces (FIG. 2A) or convex surfaces (FIG. 2B) for varied implant shapes and for reasons described below. The intervertebral discs are the largest avascular structures in the human body. The disc are substantially oval in vertical cross-section as shown in FIG. 2A, with the height of the discs increasing in varied degrees from the periphery to the center which is defined herein as a bi-convex shape. The lumbar disc of FIGS. 2A and 2B depicts a substantially convex upper (or superior) surface and inferior (or lower) surfaces that couple to the vertebral endplates 108b and 108b of the adjacent vertebrae 102a and 102b. A longitudinal ligament attaches to the vertebral bodies and to the intervertebral discs anteriorly and posteriorly, and the cartilaginous endplates 108a and 108b of each disc is attached adjacent to the bony endplates of the vertebral bodies.

A natural disc has a deceptively simple appearance, but accomplishes numerous functions. The disc's annular structure is composed of an outer annulus fibrosus AF, a constraining ring primarily composed of collagen. The gelatinous central portion of the disc, or nucleus pulposus NP, consists of proteoglycans that have highly hydrophilic branching side chains. These negatively charged regions have a strong affinity for water molecules and thus hydrate the nucleus of the disc. The hydraulic effect of the contained hydrated nucleus NP within the annulus functions as a shock absorber to cushion the spinal column from vertical forces applied to the spine.

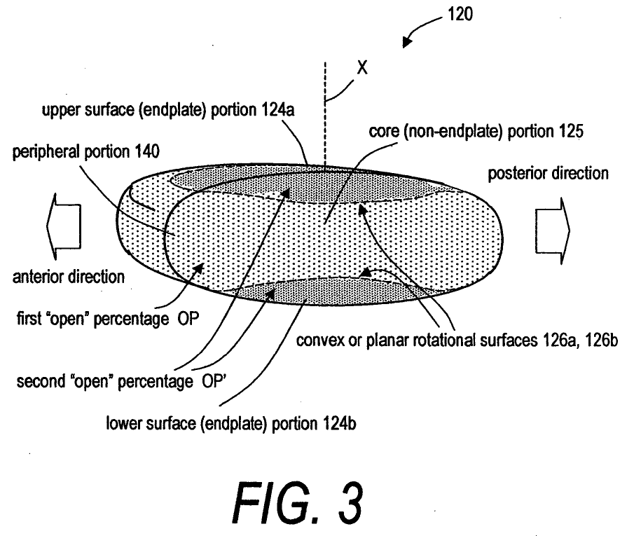

The annulus fibrosus AF also functions as a motion constraint in spinal twisting. The fibrous ring (annulus) around the nucleus has alternating collagen layers oriented at about 60° from horizontal to allow isovolumic rotation of the disc. In other words, the disc D has the ability to rotate, as well as bend, without significant change in volume—thus not affecting the hydrostatic pressure of the nucleus pulposus. As can be understood from FIGS. 3A-3B, the disc thus maintains physiologic vertebral spacing while allowing local disc compression and displacement in spinal flexion and twisting, and functions as a motion constraint during flexion by its fixation to the adjacent vertebrae.

Of particular interest to the invention, there are substantial dimensional variations in disc height dimensions, vertebral endplate concavities and posterior-to-anterior sectional shapes within the lumbar spinal segment. Typically, the intervertebral space varies in height (as defined by the opposing end plates) from posterior side to anterior side, but each disc space has a unique axial height and shape. For example, the L4-L5 intervertebral space has greater endplate concavity than the L3-L4 space. Other intervertebral spaces (e.g., the L5-S1 space) exhibit a substantial increase in axial height from the posterior to the anterior aspect thereof.

It is undesirable to be required to manufacture a single standard-sized prosthesis art for use as an implant. Thus, the replacement nucleus implant 100 corresponding to the invention is adapted for inexpensive molding and inventorying of implants in a wide range of dimensions that can be selected for a particular patient and the type of cavity or space S created to receive the implant body.

After creation of space S in the degenerated nucleus (FIGS. 1, 2A-2B), in one embodiment, the implant body 100 is assembled in-situ of first and second components indicated at 120 and 122, respectively. The first component 120 can be an open cell foam. Alternatively, the open cell body can a soft lithography micro-molded structure that is assembled layer by layer as known in the art and depicted in FIG. 3. In FIG. 3, the first component 120 comprises a biocompatible shape memory polymer (SMP) element having an open cell structure that defines a selected open volume or percentage. The second component 122 for creating the composite implant comprises an infill polymer that fills the open cells in the first component 120, and is typically infused into the first component 120 and then polymerized or cured in-situ. For example, the polymer first component is an open-cell foam composition with random, disordered open cells, or a microfabricated shape memory polymer with an ordered open cell volume. Various soft lithography methods for microfabricating an ordered, open cell implant component are known in the art.

The open cell structure is thus adapted to allow for compaction of the first component 120 when made of a shape memory polymer for low profile introduction into the space S. Of particular interest, the principal function of the first component 120 is to provide a scaffold (i) for reinforcing the implant body; (ii) for creating a rubber modulus gradient across the implant body about the axis or any radial thereof, (iii) for providing an asymmetric rubber modulus across or about radial angles of the implant body to re-distribute loads on the implant during spine flexure in an improved manner to treat specific patient disorders, and (iv) for forming a composite or matrix with the second component 122 (described next) that provides a force-control matrix that when compressed will not tend to apply forces radially outward by instead absorb deforming forces and cause the resilient implant to deflect and displacements substantially radially inwardly toward a lower modulus central portion of the implant.

The second component 122 of the implant comprises a polymer that is injectable into the space and that in-fills the first component 120 to thereafter polymerize or cure in-situ. The second component 122 can be any suitable biocompatible polymer, for example, a polymer having a suitable modulus in the class of polysiloxanes or silicones, polyurethanes, polyethers, polyamides, polyether amides, polyether esters, hydrogels or copolymers of any of the above.

The second component 122 can be selected from various inventoried polymerizable compositions to provide a selected modulus of elasticity or rubber modulus to the implant body. As will be described below, the molded or microfabricated shape memory polymer component of the invention can be easily molded and assembled to provide gradient or asymmetric open dimensions to provide an implant with precise tailored rubber moduli in various locations of the implant. For example, superior and inferior plate portions for the implant can less resilient, anterior and posterior aspects of the implant can have moduli that differ from the central portion, and the porosity of the implant or portions thereof can be optimized for fluid diffusion therethrough (to cooperate with fluid nutrient cycle). The implant also can be specifically tailored to the particular intervertebral space or the cavity created to receive the implant and the disease state of the particular patient. All these features will allow for precise tailoring of the implant for providing optimal intervertebral spacing, load distribution across the space, kinematics and endurance.

In an exemplary embodiment, the implant body 100 (FIGS. 1 and 3) of the invention is adapted to improve disc function by enhancing and maintaining vertebral spacing and at the same time functioning as a shock absorber for vertical loads. The disc implant 100 is less suited for acting as a motion constraint is since it is not adapted for fixation to the vertebral endplates 108a and 108b. In this embodiment, the natural annulus AF is retained as the primary motion constraint means and stability means together with the anterior and posterior ligaments that couple the adjacent vertebrae. For this reason, the implant 100 is not designed in cross-section to exactly resemble a natural disc, and in one embodiment of FIG. 3 can have upper and lower surface portions (or endplates) 124a and 124b that have a substantially high rubber modulus when compared to the modulus of the non-endplate or core portion 125 to define planar or concave rotational surfaces indicated at 126a and 126b in FIGS. 3 and 5 (concave in this embodiment). The gradient in modulus between the vertebral endplates 108a and 108b and the non-endplate, core portion 125 of the implant, it is believed will prevent implant displacement or slippage to prevent undue stress on the natural annulus AF—with the result being improved overall stability of the treated vertebral segment. At the same time, a concave rotational surfaces 126a and 126b as in FIG. 3 can provide improved hydraulic-like cushioning under spinal flexion by making the implant have a lower effective rubber modulus at the central portion 130 of the implant and a higher effective modulus at the periphery 140 simply by the thickness of the non-endplate portion 125. As will be described below, the radial gradient of core or non-endplate portion 125 of the implant 100 can prevent the adjacent vertebrae from rotating around the centerpoint or the implant-as may the case with other prior art nucleus implants.

FIGS. 3 and 5 illustrate sectional views of first component 120 of the implant with FIG. 5 illustrating the component elements de-mated to depict the method of making and assembling the component 120 from core 125 and end portions 124a and 124b. In this embodiment, the SMP foam of core 125 defines a first open percentage OP for receiving the in-fill polymer. The open cell end portions 124a and 124b define a second open percentage OP′ for receiving the in-fill polymer, which is substantially less the first open percentage. The core and endplate sub-components 125, 124a and 124b are bonded together by any suitable porous bonding means, for example, thermal bonding, or chemical adhesive bonding.

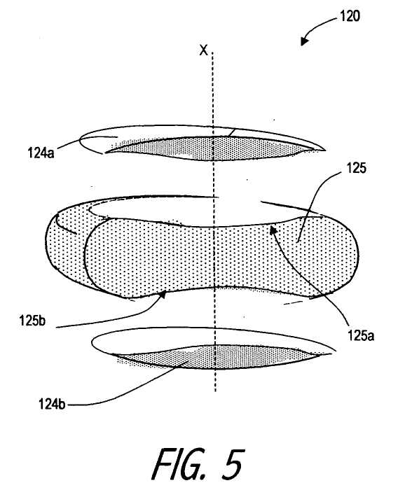

FIG. 6 illustrates an alternative first component 150 of a disc implant corresponding to the invention (showing the inferior half with a similar superior half not shown). It can be seen that an annular or donut shaped element 144 is molded to be assembled with the core 125 and end portions 124a and 124b. Each element can have a selected open percentage OP, OP′ and OP″ to thereby tailor the performance of the implant under spinal flexion and twisting.

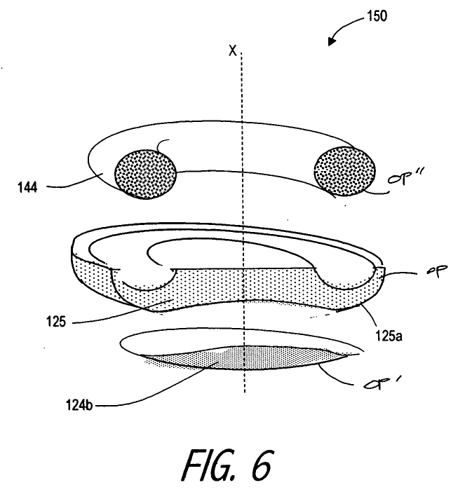

FIG. 7 illustrates another alternative first component 160 of a disc implant, again showing the inferior half with a cooperating superior half not shown. This implant component has two cooperating annular elements 144 and 145 that each define a different selected open percentage to thereby great a gradient modulus across the peripheral portion 140 of the implant body when infused with the second polymer component 122 that is polymerized after introduction. It should be appreciated that the scope of the invention includes any number of molded and cooperating elements with different open percentages to create any desired gradient in rubber modulus across portions of the implant.

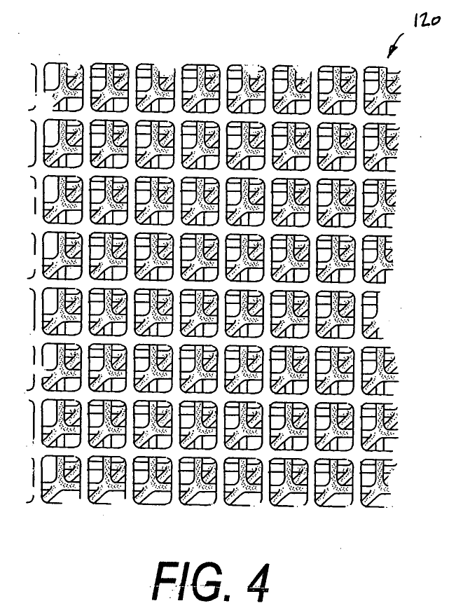

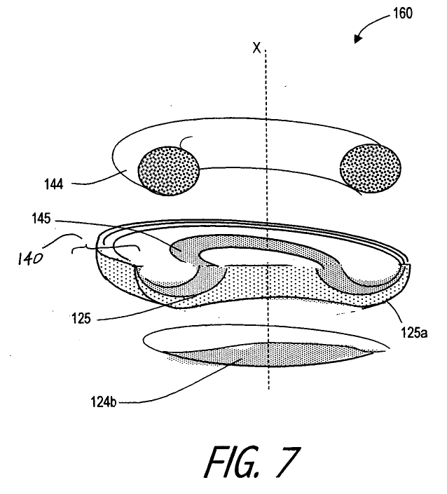

FIG. 8 illustrates another exemplary first component 170 of a disc implant (cooperating superior half not shown). The implant component of FIG. 8 is a preferred type of embodiment that is likely to be the most commonly used, wherein the body has posterior and anterior interior elements 154 and 155 that are adapted to tailor the compression response characteristics of the prosthesis in different manners for spine flexion and extension. The posterior and anterior open cell elements 154 and 155 can have different open percentages or they can have similar open percentages. Any number of cooperating nested together elements can be used to create the desired modulus gradient. Further, it can be seen that the posterior-to-anterior vertical sectional shape is tapered to fully occupy the shape of the space typically created. The implant can be provided with or without endplate portions 124a and 124b. Any implant embodiment with endplate portions 124a and 124b as in FIGS. 4 and 5-8 is typically adapted for spaces S as in FIGS. 1 and 2A-2B. The implants also can be used with BMPs in the surfaces for enhancing bone ingrowth into the surface regions for fusing the implant surface to the two vertebral endplates—each being thin cartilage layers overlying cortical bone and the vascularized cancellous interior bone.

In sum, the modulus of the polymer of the first component 120 is higher than the cured modulus of the in-fill polymer second component 122 so that it can be easily understood that the matrix within the portion of the implant having a lesser open percentage will have a matrix modulus that is defined by the combination of the webs of the first component and the in-fill polymer 122. Any void portion of the first component or that has a very high open percentage will have a modulus that is effectively defined by the polymer second component 122 alone. By this means, it can be seen that the invention provides an implant that defines a very modulus of elasticity along a radial of the implant body 100.

Thus, the implant body 100 comprises an in-situ assembled elastic composite material that can be engineered to provide (i) selected resistance to compression during spine flexion and extension to maintain vertebral spacing, and (ii) resistance to lateral outward displacement of any compressed portion of the implant body during spine flexion to again control and maintain vertebral spacing particularly at the periphery of the vertebrae that are urged closer together on spine flexion. In this aspect of the implant's functionality, the network or webs of the first component serve as reinforcing to limit lateral outward displacement of any peripheral portion of the implant.

The embodiments of FIGS. 4 and 5-8 also may be assembled with a reinforcing component comprising any synthetic non-stretch fibers or filaments of any length, (Kevlar, etc) to limit deformation of the first component 120.

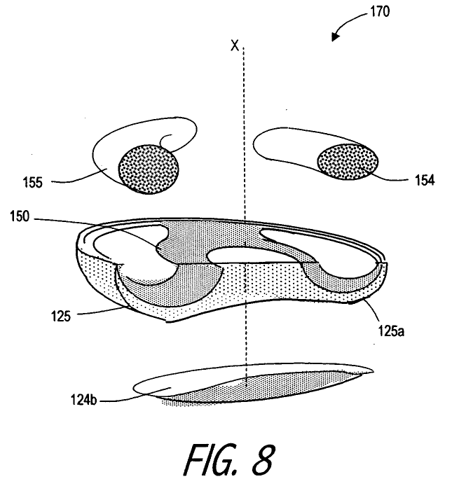

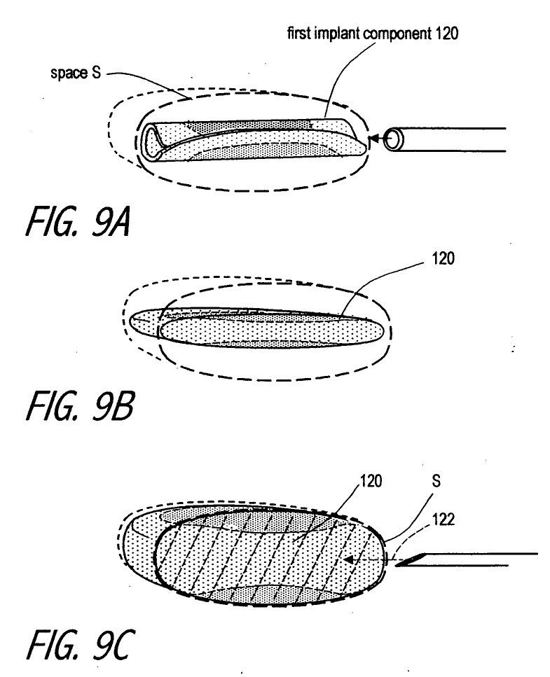

In any embodiment of FIGS. 4 and 5-8, the first component 120, 150, 160 or 170 is of a shape memory polymer foam or microfabricated open cell structure that can be compacted. For example, as illustrated in FIGS. 9A-9C, the first component 120 can be introduced in a highly compacted state and then allowed to expand to comprise a stretchable or substantially non-stretchable body that occupies the space S as in FIG. 1. The cell network of the polymer body 120 further acts as a reinforcing element in an infused polymer matrix.

As background, the class of shape memory polymers (SMPs) comprises a type of co-polymer that consists of a hard segment and a soft segment each having a different glass transition temperature. One segment has a glass transition temperature ranging between about 35° C. and 80° C. at which the shape memory polymer changes from a first dimension or volume to a second dimension or volume. For example upon deployment in tissue, one segment of the polymer can have a glass transition temperature of about 35° C. to 37° so that body temperature causes the implant to move from an initial compacted position to an expanded position.

The implant component 120 of FIG. 9A can preferably expands at body temperature as depicted in FIGS. 9B-9C. Alternatively, the component 120 can carry any suitable biocompatible material that cooperates with photonic energy, electrical energy or magnetic energy to elevate its temperature. Light sources, Rf sources and magnetic emitters are known and can be used to deliver energy to the implant, e.g., as disclosed in the author's U.S. patent application Ser. No. 09/473,371 filed Dec. 27, 1999 (now U.S. Pat. No. 6,306,075), incorporated herein by reference. The detail of the energy source need not be further described herein. The application of energy from any source can be used with an implant that is designed to have a transition temperature anywhere above about 37° C.—for example, in a range extending from about 370 to 80° C. The step of elevating the temperature of an implant component is typically performed immediately after implantation, but the scope of the invention includes using magnetic resonant means, for example, at a later time to expand the implant component or alter the component's ability to diffuse water, or alter other functional parameters.

As will be described below, in another embodiment, the implant body can carry a biodegradable or bioresorbable polymer as is known in the art and is among the polymer materials listed previously. Further, such polymer can thus allow for porosities that allow for tissue ingrowth. Further, the biodegradable or bioresorbable polymer can be triggered by to degrade, or enhance degradation, by the magnetoresonant means described in U.S. Pat. No. 6,306,075.

The shape memory polymers (SMPs) used in the first component 120 of FIGS. 4-8 demonstrate the phenomena of shape memory based on fabricating a segregated linear block co-polymer, typically of a hard segment and a soft segment. The shape memory polymer generally is characterized as defining phases that result from glass transition temperatures in the hard and a soft segments. The hard segment of SMP typically is crystalline with a defined melting point, and the soft segment is typically amorphous, with another defined transition temperature. In some embodiments, these characteristics may be reversed together with the segment's glass transition temperatures.

In one embodiment, when the SMP material is elevated in temperature above the melting point or glass transition temperature of the hard segment, the material then can be formed into a memory shape. The selected shape is memorized by cooling the SMP below the melting point or glass transition temperature of the hard segment. When the shaped SMP is cooled below the melting point or glass transition temperature of the soft segment while the shape is deformed, that temporary shape is fixed. The original shape is recovered by heating the material above the melting point or glass transition temperature of the soft segment but below the melting point or glass transition temperature of the hard segment. (Other methods for setting temporary and memory shapes are known which are described in the literature below). The recovery of the original memory shape is thus induced by an increase in temperature, and is termed the thermal shape memory effect of the polymer. The transition temperature can be body temperature or somewhat below 37° C. in many embodiments-or a higher selected temperature when the implant body is adapted to cooperate with magnetic responsive particles or chromophores in the polymer that cooperate with a remote energy source.

Besides utilizing the thermal shape memory effect of the polymer, the memorized physical properties of the SMP can be controlled by its change in temperature or stress, particularly in ranges of the melting point or glass transition temperature of the soft segment of the polymer, e.g., the elastic modulus, hardness, flexibility, and permeability. The scope of the invention of using SMPs in implants extends to the control of such physical properties within the implant for numerous therapeutic applications.

Examples of polymers that have been utilized in hard and soft segments of SMPs include polyethers, polyacrylates, polyamides, polysiloxanes, polyurethanes, polyether amides, polyether esters, and urethane-butadiene copolymers. See, e.g., U.S. Pat. No. 5,145,935 to Hayashi; U.S. Pat. No. 5,506,300 to Ward et al.; U.S. Pat. No. 5,665,822 to Bitler et al.; and U.S. Pat. No. 6,388,043 to Langer et al, all of which are incorporated herein by reference. SMPs are also described in the literature: Ohand Gorden, Applications of Shape Memory Polyurethanes, Proceedings of the First International Conference on Shape Memory and Superelastic Technologies, SMST International Committee, pp. 115-19 (1994); Kim, et al., Polyurethanes having shape memory effect, Polymer 37(26):5781-93 (1996); Li et al., Crystallinity and morphology of segmented polyurethanes with different soft-segment length, J. Applied Polymer 62:631-38 (1996); Takahashi et al., Structure and properties of shape-memory polyurethane block copolymers, J. Applied Polymer Science 60:1061-69 (1996); Tobushi H., et al., Thermomechanical properties ofshape memory polymers of polyurethane series and their applications, J. Physique IV (Colloque C1) 6:377-84 (1996)) (all of the cited literature incorporated herein by this reference).

Of particular interest, the use of an open structure of a shape memory polymer provides several potential advantages in implants, for example, very large shape recovery strains are achievable, e.g., a substantially large reversible reduction of the Young's Modulus in the material's rubbery state; the material's ability to undergo reversible inelastic strains of greater than 10%, and preferably greater that 20% (and up to about 200%-400%); shape recovery can be designed at a selected temperature between about 30° C. and 45° C., and injection molding is possible thus allowing complex shapes. These polymers demonstrate unique properties in terms of capacity to alter the material's water or fluid permeability, thermal expansivity, and index of refraction. However, the material's reversible inelastic strain capabilities leads to its most important property—the shape memory effect. If the polymer is strained into a new shape at a high temperature (above the glass transition temperature Ts) and then cooled it becomes fixed into the new temporary shape. The initial memory shape can be recovered by reheating the foam above its Tₛ. The shape memory foams are of particular interest for various implants because they provide even lower density than solid SMPs.

FIGS. 9A-9D schematically illustrate the method of implanting any Type “A” embodiment of FIGS. 1,2 and FIGS. 4-8. In FIG. 9A, the first component 120 in a flattened and rolled cylindrical configuration is being deployed in space S. It should be appreciated that the implant can be compacted or crushed radially to comprise a small diameter cylinder. FIG. 9B shows the first component 120 in the process of expanding in the space S. FIG. 9C schematically shows the step of infusing the first component 120 with an infill polymer or second component 122 that is thereafter cured in-situ to provide the final implant configuration. After the polymer 122 cures within the open portion of the SMP first component 120, the matrix of the polymers will result in the nucleus implant of FIG. 1 with the selected variable localized rubber moduli or durometers across the implant.

The implant as described above is expected to provide a certain degree of porosity to allow fluid diffusion therethrough. The native disc is adapted for such fluid diffusion to support metabolism within the disc tissue. Such fluid diffusion is caused by compression and decompression of the disc resulting in inflows of nutrients and outflows of waste. In essence, differential osmotic pressures induce such fluid diffusion in the disc. Typically, in the patient's waking hours, when intradisc pressure will increase due to the forces of gravity and loads on the spine. Thus, nucleus pulposus can lose as much as 20 percent of its water content to wash out the byproducts of anaerobic metabolism and the disc will actually become thinner. At rest during the night, the disc will rehydrate with nutrients in effect creating an osmotic pump system that enables normal disc metabolism. The osmotic forces and fluid diffusion must be accommodated with the nucleus implant of the invention. It is believed that any artificial disc that allows for fluid diffusion can accommodate the normal osmotic flows and prevent the accumulation of waste products and inflammatory compositions in the disc space. To enhance fluid diffusion, the infill polymer 122 can in part comprise a hydrogel that allows for such fluid diffusion.

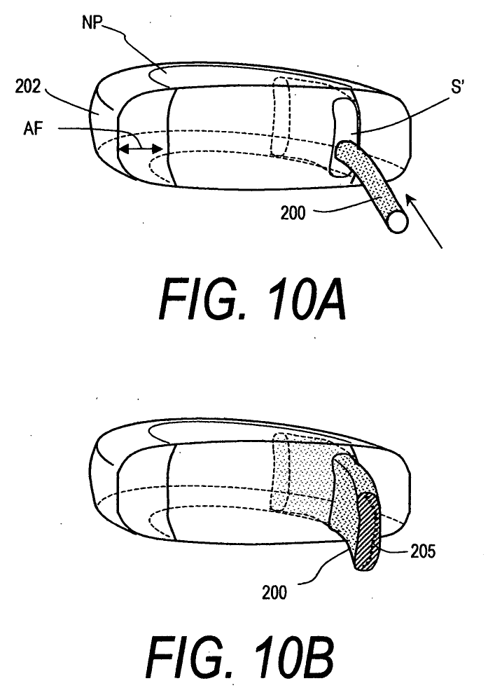

FIGS. 10A-10B illustrate another spine implant body of a shape memory polymer. The device or body comprises an annulus reinforcing structure indicated at 200. In FIG. 10A, the disc 202 has a space S′ created about the inner surface of the annulus AF by a cutting or ablation device. The implant 200 is an open cell shape memory polymer as describes above that can be compacted for introduction into the space S′ (cf. FIG. 9A) wherein it expands to occupy the space. Of particular interest, the implant body 200 carries a reinforcing structure 205 of any suitable material (e.g., NiTi, Kevlar etc.). One advantage of the invention relates to the fact that the SMP polymer that will expand and engage the inner surface of the annulus AF about space S′—as opposed to other reinforcing approaches that can only partly occupy a necessarily irregular space. When such an irregular space is loaded and collapses, there may be even greater pressure on the targeted treatment region—such as a weakened or herniated region. Further, the SMP can be provided to have a selected modulus, for example to match the natural disc. The polymer can carry any pharmacological agent to enhance disc regeneration. The annulus reinforcing body 200 can be provided in any shape and dimension to occupy any space that is created by a cutting or ablation device.

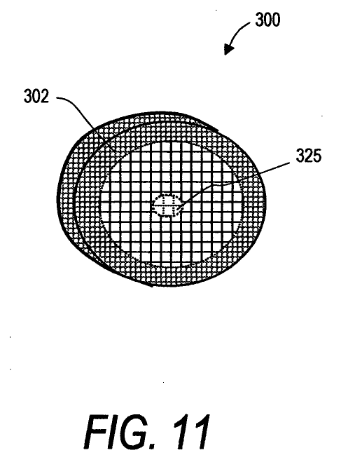

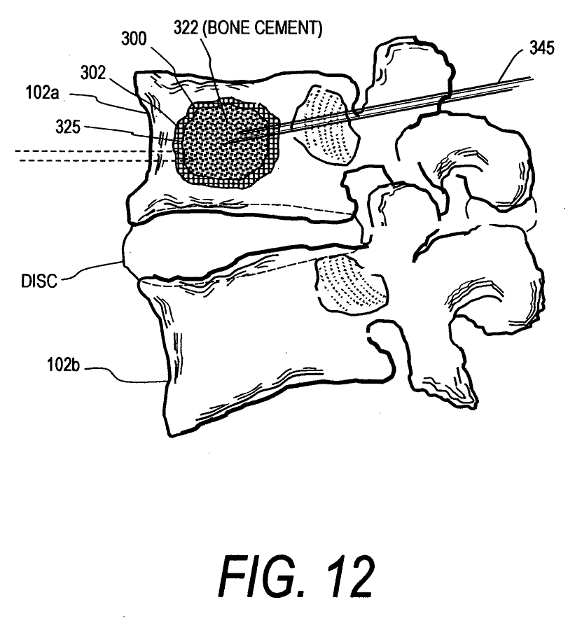

FIG. 11 illustrates a schematic sectional view of an alternative shape memory polymer implant 300 that is adapted for treating a compression fracture. The interior region 302 of open cell implant is adapted to substantially seal and contain an in-situ hardenable bone cement such as PMMA. In FIG. 12, a spine segment is shown with vertebra 102a having a compression fracture, resulting in a collapse in vertebral height. In FIG. 12, the height is increased and space S is created in cancellous bone 302. Such a space S as indicated in FIG. 12 can be created by compaction with a balloon, other compaction means or the space can be cut or reamed out as is known in that art, or a combination of compaction and material removal. The space S can comprise a single cavity or a multiplicity of spaces.

An open cell SMP body 300 as in FIG. 11 for treating cancellous bone differs somewhat in function than the previous embodiments. The open cell body is of an elastomeric polymer having a suitable modulus to allow its networked cell walls to stretch substantially to compact into the extremities of a cavity created in a bone. The open cell body 300 is similar to previous embodiments in that it is adapted for compaction to allow its introduction through a small diameter introducer. The implant 300 in similar to previous embodiments in that the open cell body is adapted to thereafter expand to occupy all extremities of the space or cavity. Implant 300 differs from previous embodiments in that its open cell walls 302 have a selected open cell dimension that substantially prevents diffusion of a hardening polymeric bone cement. Thus, the implant cell wall network is adapted to deform outwardly upon injection of a relatively high viscosity bone cement component 322, rather than functioning as a reinforcing network within the injected polymer as in previous embodiments. In a preferred embodiment, the open cell body has a gradient polymer networked structure with a greater open cell volume, or an open space in a center of the implant body 300. In a preferred embodiment, a central region of the implant also carries at least one radiovisible marker 325 (e.g., any suitable radiopaque material such as a platinum element). In any open cell body 300, the polymer has a modulus of less than about 1 MPa, and more preferably less than about 500 KPa to provide the required elasticity.

In use, the implant body 300 is introduced into space in the bone (FIG. 12) and a distal tip of the bone cement injector 345 is localized in the region of radiovisible marker 325. Thereafter, injection of a bone cement such as PMMA will create a composite implant structure comprising a solid polymeric core portion and a peripheral region of compacted elastomeric body wall 302 that is pressed outwardly to the extremities of the space. Of particular interest, the compacted body will have a multiplicity of cells wall network layered over on another to create a substantially fluid impermeable layer about the core portion of a bone cement. This use of the open cell polymeric implant body 300 is thus useful in containing the bone cement 322 in a substantially fluid impermeable outer layer to prevent migration of the bone cement composition into the blood stream or into contact with nerves. The use of such an open cell polymer body solves a problem associated with prior art procedures wherein PMMA bone cement is injected directly into a bone cavity and its components can cause damage to nerves or can enter the circulatory system and have serious consequences on the patient's health. While described in conjunction with a compression fracture of vertebra, it should be appreciated that the implant for sealing the extremities of a bone cavity from bone cement migration extends to similar uses in all bones such as the tibia and femur that are often treated with bone cement.

The scope of the invention thus comprises any open cell or open volume elastomeric body dimensioned for implantation in a bone cavity for cooperating with an injected bone cement to provided a substantially impermeable surface. In another embodiment, the implant body can be provided with an open interior volume by means of electrospinning polymer fibers or filaments that range in cross-section from about 100 nm to 5 microns. Such electrospun fibers then can be formed into a shape open volume mat or monolith and used as described above. In particular, such an implant body of electrospun fibers would be suitable for creating an annulus reinforcing structure as in FIGS. 10A-10B and in creating a bone cement sealing structure as in FIGS. 11 and 12.

Although particular embodiments of the present invention have been described above in detail, it will be understood that this description is merely for purposes of illustration. Specific features of the invention are shown in some drawings and not in others, and this is for convenience only and any feature may be combined with another in accordance with the invention. Further variations will be apparent to one skilled in the art in light of this disclosure and are intended to fall within the scope of the appended claims.

What is claimed is:

1. A spine implant comprising a body for dimensioned for engaging spine structure including an open cell shape memory polymer.

2. A spine implant as in claim 1 wherein the open cell shape memory polymer has a gradient in open cell volume.

3. A spine implant as in claim 1 wherein the open cell shape memory polymer is a soft lithography microfabricated structure.

4. A spine implant as in claim 1 wherein the open cell shape memory polymer is a foam.

5. A spine implant as in claim 1 wherein the body has a first unstressed state and is releasably maintainable in a second stressed state having a compacted shape.

6. A spine implant as in claim 5 wherein the body is transformable to the first unstressed state from the second stressed state in response to a controllable stimulus.

7. A spine implant as in claim 6 wherein the stimulus is temperature.

8. A spine implant as in claim 7 wherein the stimulus is temperature between about 35° C. and 120° C.

9. A spine implant as in claim 5 wherein the shape memory polymer is the first unstressed state has a modulus of ranging between 10 KPa and 5 MPa.

10. A spine implant as in claim 1 wherein the body further includes reinforcing fibers.

11. A spine implant as in claim 5 wherein the body is dimensioned in its unstressed state for occupying a space selected from the class consisting of a disc nucleus space, a space about the interior of a disc annulus, and a space in a bone.

12. A spine implant as in claim 1 further comprising an in-situ polymerizable composition for introducing into an interior of the body for creating a composite implant.

13. A spine implant as in claim 1 further comprising a radiovisible element within the body.

14. A method of treating an orthopedic abnormality comprising:
(a) providing an implant body of an open cell shape memory polymer that is releasably maintained in a compacted, stressed state;
(b) introducing the implant body into a space within orthopedic structure; and
(c) causing the implant body to transform in shape to an expanded, unstressed state.

15. A method of treating an orthopedic abnormality as in claim 14 wherein the implant body expands to position reinforcing fibers about the interior of a disc annulus.

16. A method of treating an orthopedic abnormality as in claim 14 wherein the implant body expands to occupy a disc nucleus space, and further comprises the step of infilling open cell body with an in-situ polymerizable composition.

17. A method of treating an orthopedic abnormality as in claim 14 wherein the implant body expands to occupy a space in a bone, and further comprises the step of introducing a bone cement into an interior of the implant body.

18. A method of treating an orthopedic abnormality as in claim 17 wherein the implant body forms a substantially fluid impermeable layer around the bone cement.

19. A method of making an orthopedic implant device comprising microfabricating a polymeric body having a substantially open interior volume by microfabrication means selected from the class consisting of soft lithography means, electrospinning means and foaming means.

20. A method of making an orthopedic implant device as in claim 19 wherein the body includes a shape memory polymer.
